(19) United States
(12) Patent Application Publication
(10) Pub. No.: US 2008/0132776 A1
(43) Pub. Date: Jun. 5, 2008

(54) MAGNETIC RESONANCE METHOD AND APPARATUS FOR SELECTIVE EXCITATION OF NUCLEAR SPINS

(76) Inventor: Uwe Boettcher, Uttenreuth (DE)

Correspondence Address: SCHIFF HARDIN, LLP;PATENT DEPARTMENT, 6600 SEARS TOWER, CHICAGO, IL, 60606-6473, US

(21) Appl. No.: 11/942,887

(22) Filed: Nov. 20, 2007

(30) Foreign Application Priority Data
Nov. 20, 2006 (DE) 10 2006 054 599.0

(51) Int. Cl. G01V 3/14; A61B 5/055

(52) U.S. Cl. 600/410; 324/307

(57) ABSTRACT

In a method and magnetic resonance apparatus for selective excitation of nuclear spins of an examination region in an examination subject using at least one radio-frequency excitation pulse and using slice-selective magnetic fields, the slice-selective magnetic field gradients are selected dependent on the position of the examination region relative to at least one structure surrounding the examination region.

### BACKGROUND OF THE INVENTION

1. Field of the Invention

The present invention concerns a method for selective excitation of nuclear spins of an examination region in an examination subject (as used in magnetic resonance spectroscopy) as well as a magnetic resonance apparatus for implementation of such a method.

2. Description of the Prior Art

Specific chemical compounds can be detected in a spatially selective, non-invasive and non-destructive manner using magnetic resonance spectroscopy (designated in the following as MRS, MR for magnetic resonance). In healthy tissue the metabolic products (metabolites) that can be detected by means of MRS exist in generally known equilibrium concentrations typical to the tissue. Stress, function disruptions or illnesses can lead to shifts of the metabolite concentrations. Such concentration changes can be detected with MRS, which is why MRS is an important method for in-vitro and in-vivo examination of the cell metabolism of tissues and organs.

MRS is based on the same basic principles as magnetic resonance imaging (MRI). Significantly simplified magnetic resonance signals in a subject to be examined (which are subsequently detected, are excited by magnetic fields of different strengths and spatial and temporal characteristics. In MRI, information about the spatial distribution of the excited nuclear spins can be obtained from the acquired measurement data, from which images of the subject to be examined can be produced. In MRS, information about the concentration of specific metabolites in a region to be examined can also be obtained from the spectral distribution of the measured signal.

To produce the magnetic resonance signals, the nuclear spins to be excited are initially positioned in a comparably strong external, static magnetic field B0 (field strengths of typically 0.2 Tesla up to 7 Tesla and more) such that the nuclear spins align in the external magnetic field (also designated as a basic magnetic field). The deflection of the aligned nuclear spins from the stable position is achieved by means of radio-frequency (RF) energy. The required energy or frequency ω₀ of the waves is thereby precisely established for each nucleus; it is determined by a nuclear property (the “gyromagnetic ratio” γ) and by the strength of the applied magnetic field B₀:ω₀=γB₀. If the RF waves are at a frequency that is only barely off resonance, no excitation is possible.

This fact can be utilized in order to excite (resonate) only specific, spatially localized nuclear spins within a sample. For example, a spatially-localized excitation of nuclear spins (i.e. a volume-selective excitation) is frequently used in MRS in order to examine a defined examination region in a targeted manner. In this defined examination region ideally only nuclear spins of the examination region are excited and their emitted nuclear magnetic resonance signals are measured and evaluated. For this purpose, magnetic field gradients are superimposed on the static magnetic field so that the resulting magnetic field strength varies spatially. Skillful superimposition of magnetic field gradients during the irradiation with radio-frequency energy, it can achieve the results of only nuclear spins in a predefined examination region being excited to resonance.

The dependency of the resonance frequency of the nuclear spins on the applied magnetic field, however, is also due to the fact that nuclear spins have a different resonance frequency when they are located in different chemical compounds and/or a different chemical environment, since a different shielding of the static magnetic field exists at the site of the nucleus dependent on the chemical compounds. This shift of the resonance frequency of nuclear spins is designated as a “chemical shift”. For example, protons in fat and in water exhibit a difference of approximately 3.7 ppm (parts per million) at the resonance frequency.

Such chemical shifts form the basis for MRS since the signals emitted by excited nuclear spins due to the chemical shift exhibit different frequencies that are embodied in the frequency spectrum of the measured signal.

The chemical shift, however, also leads to problems in the targeted excitation of a region to be examined using magnetic field gradients that are superimposed on the static magnetic field. Due to the chemical shift, the excited volumes for various metabolites are spatially offset from one another. The spatial shift of these volumes relative to one another depends on the direction of the applied magnetic field gradients. This means that (given the presence of different metabolites) nuclear spins outside of a desired examination region may be excited as well.

This is particularly problematic when nuclear spins outside of the desired examination region are excited that exit a very intensive signal. For example, in proton spectroscopy, a region to be examined may border fatty tissue. When magnetic field gradients are now applied so that predominantly protons in the examination region are excited, in spite of this protons of the fatty tissue can be excited as well since the protons of the fatty tissue exhibit a slightly different resonance frequency and therefore the excitation region for protons with a chemical shift typical for fatty tissue does not coincide with the examination region to which the excitation frequencies of the radio-frequency pulses have been tuned. The signal of these unwanted excited protons in the measured spectra of the examination region can cause an evaluation of the spectra to no longer be possible, since weaker signals of interest can be superimposed and no longer separated from the unwanted signals. This effect plays an increasing role at higher field strengths, since with such higher field strength a relatively large shift of the excitation regions occurs for metabolites with different resonance frequencies, since the frequency differences between the metabolites increase with the field strength.

A conventional approach to remedy this problem has been to modify the excitation frequency such that the excitation region for nuclear spins whose resonance frequency is characterized by a specific chemical shift (for example by the chemical shift of fat) coincides with the examination region. The excitation frequency, however, then lies at the edge of the spectrum, and the chemical shift artifact is greater for the metabolites of interest.

### SUMMARY OF THE INVENTION

An object of the invention to provide a method for selective excitation of nuclear spins in an examination region with which the subsequently acquired measurement signal is contaminated to only a slight extent by resonance signals of nuclear spins that lie outside of the examination region. Furthermore, it is an object of the invention to provide a magnetic resonance apparatus with which nuclear spins in an examination region can be selectively excited such that the subsequently acquired measurement signal is contaminated to only a slight extent by signals of nuclear spins that lie outside of the examination region.

In the inventive method for selective excitation of nuclear spins of an examination region in an examination subject using at least one radio-frequency excitation pulse and using slice-selective magnetic fields, the slice-selective magnetic field gradients are selected dependent on a position of the examination region relative to at least one structure surrounding and in particular adjoining the examination region. In the inventive method, the slice-selective magnetic field gradients are no longer selected dependent solely on the position of the examination region (as was previously typical) but also are selected with consideration of the position of the examination region relative to surrounding structures. This dependency now enables an unwanted excitation of nuclear spins in the surrounding structure (which would otherwise be possible due to the chemical shift of the resonance frequencies of nuclear spins of the surrounding structure) to be avoided.

In a preferred embodiment, the slice-selective magnetic field gradients are selected such that an excitation region of nuclear spins whose resonance frequency is characterized by a specific chemical shift lies further removed from the surrounding structure than the examination region. Because the excitation region of nuclear spins of a specific chemical shift is further removed from the surrounding structure than the examination region itself, an excitation of nuclear spins in the surrounding structure with the specific chemical shift can be minimized or even prevented in a safe manner.

The specific chemical shift advantageously corresponds to the chemical shift of nuclear spins in fatty tissue. A case frequently occurring in MRS examinations of a human body, namely that nuclear spins of fatty tissue that surrounds the examination region are also excited as well in an undesirable manner by the excitation of nuclear spins in an examination region, can be avoided in this way in a simple and safe manner.

In another embodiment, the orientation of the slice-selective magnetic field gradients is selected dependent on the position of the examination region relative to the surrounding structure. By the selection of the orientation of the slice-selective magnetic field gradients, the position of the examination region relative to surrounding structures can be taken into account in a particularly simple manner without the magnetic field gradients that are used having to be recalculated in a complicated manner.

In an embodiment that is particularly simple, the orientation of the slice-selective magnetic field gradients is determined by selection of the polarity of the magnetic field gradients of at least one gradient coil. This embodiment also enables a particularly simple consideration of the position of the examination region relative to surrounding structures without elaborate recalculation of the magnetic field gradients.

In another embodiment of the method, the examination region is established using an overview image. This enables a simple adaptation of an MRS examination to the present anatomical relationships.

In a further embodiment, the selection of the slice-selective magnetic field gradients ensues automatically. This embodiment is primarily suitable in MRS examinations given known anatomical relationships in which the position of an examination region relative to surrounding structures is known, such that the quality of results given such examinations can be automatically improved.

In another embodiment, the selection of the slice-selective magnetic field gradients ensues by interaction with a user. This embodiment variant is particularly suitable given variable anatomical relationships in which an automatic selection of the magnetic field gradients sometimes does not lead to desired results. Through the interaction, a user can check whether a specific selection of the magnetic field gradients correctly takes into account the position of surrounding structures and can modify the selection of the magnetic field gradients if necessary. The flexibility of and the range of use of the method are thereby increase.

In a preferred embodiment, the user is supported in the selection of the slice-selective magnetic field gradients by the examination region being presented in an overview image together with a presentation of the excitation region of nuclear spins whose resonance frequency is characterized by a specific chemical shift. The user thus can graphically check whether the position of surrounding structures is correctly taken into account by the selection of the magnetic field gradients. In this way a user is supported in an effective and simple manner in the selection of the slice-selective magnetic field gradients.

The inventive magnetic resonance apparatus has a control computer that is fashioned for implementation of a method described above as well as all embodiments.

### BRIEF DESCRIPTION OF THE DRAWINGS

FIG. 1 schematically illustrates the basic design of a magnetic resonance apparatus.

FIG. 2 illustrates basic steps of an embodiment of the inventive method.

FIG. 3 shows a phantom presented using three overview images orthogonal to one another.

FIG. 4 shows the time curve of applied magnetic field gradients relative to the volume-selective excitation.

FIG. 5 shows the time curve of applied magnetic field gradients with partially reversed polarity.

FIG. 6 shows a frequency spectrum of the measured signal with contamination by protons of a fatty substance.

FIG. 7 shows a further frequency spectrum of a measured signal with distinctly reduced contamination.

### DESCRIPTION OF THE PREFERRED EMBODIMENTS

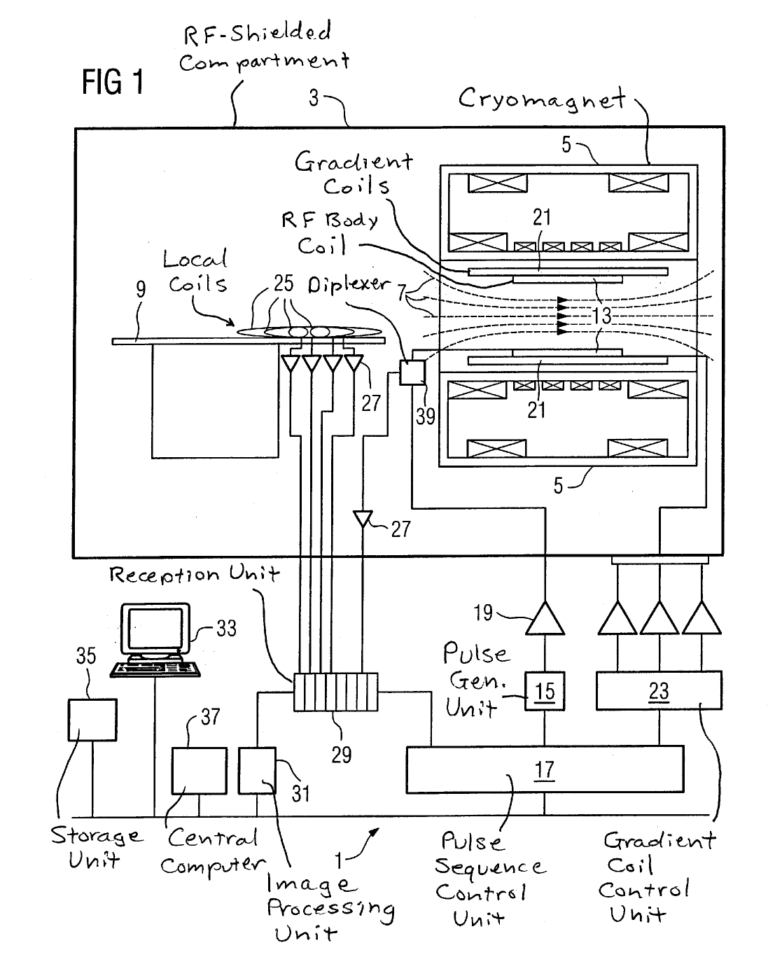

FIG. 1 schematically shows the basic design of a magnetic resonance apparatus 1. The components of the magnetic resonance apparatus 1 with which the actual measurement is implemented are located in a radio-frequency-shielded measurement compartment 3. In order to examine a body by means of magnetic resonance, various magnetic fields tuned as precisely as possible to one another in terms of their temporal and spatial characteristics are radiated at the body.

A strong magnet, typically a cryomagnet 5 with a tunnel-shaped opening, generates a strong, static basic magnetic field 7 that is typically 0.2 Tesla to 7 Tesla and more and that is largely homogeneous within a measurement volume. A body (not shown here) to be examined is supported on a patient bed 9 and is positioned in the basic magnetic field 7 (more precisely in the measurement volume).

The excitation of the nuclear spins of the body ensues by magnetic radio-frequency excitation pulses that are radiated from a radio-frequency antenna (shown here as a body coil 13). The radio-frequency excitation pulses are generated by a pulse generation unit 15 that is controlled by a pulse sequence control unit 17. After amplification by a radio-frequency amplifier 19, they are conducted to the radio-frequency antenna 13. The radio-frequency system shown here is only schematically indicated. Typically multiple radio-frequency antennas are used in a magnetic resonance apparatus 1 and to some extent more than one pulse generation unit 15 and more than one radio-frequency amplifier 19 are also used.

Furthermore, the magnetic resonance apparatus 1 has gradient coils 21 with which gradient fields for selective slice or volume excitation and for spatial coding of the measurement signal are radiated given a measurement. The gradient coils 21 are controlled by a gradient coil control unit 23 that, like the pulse generation unit 15, is connected with the pulse sequence control unit 17.

The signals emitted by the excited nuclear spins are received by the body coil 13 and/or by local coils 25, are amplified by associated radio-frequency preamplifiers 27, and are further processed and digitized by a reception unit 29. The reception coils can also include a number of coil elements with which magnetic resonance signals are simultaneously received.

In the case of a coil that can be operated both in transmission mode and in reception mode (such as, for example, the body coil 13), the correct signal relaying is regulated by an upstream transmission-reception diplexer 39.

An image processing unit 31 generates from the measurement data an image that is presented to a user at a control console 31, or that is stored in a storage unit 35. A central computer 37 controls the individual system components. The computer 37 and the further components are fashioned to implement the inventive method.

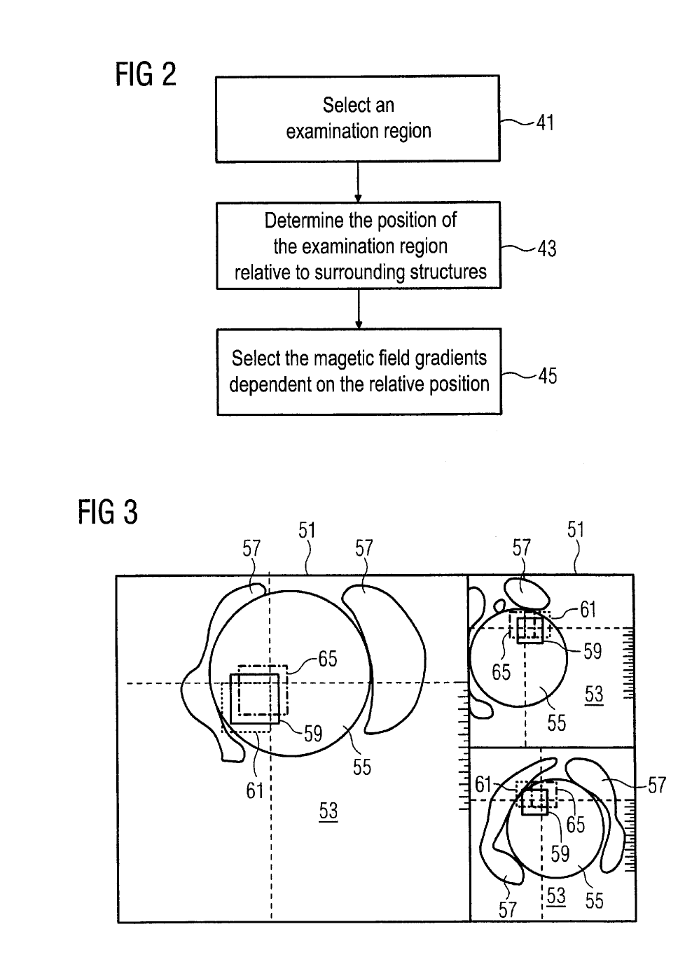

An explanation of basic method steps of a preferred embodiment of the inventive method now ensues using FIG. 2. In a first method step 41 an examination region in a subject to be examined is selected. This examination region is to be examined by magnetic resonance spectroscopy, whereby in that nuclear spins of the examination region are excited in a targeted manner and their emitted measurement signals are evaluated.

The selection of the examination region can ensue, for example, using an overview image by user interaction who can mark the examination region in the overview image. Given known anatomical relationships and standardized examinations, however, the selection of the examination region can also ensue automatically, possibly in connection with known pattern recognition algorithms or segmentation algorithms.

Using the spatial position of the examination region in the examination subject, radio-frequency excitation pulses and magnetic field gradients can now be tuned to one another such that predominantly only those nuclear spins that are located in the examination region are excited to resonance.

While in conventional methods the magnetic field gradients were selected as being suitable based solely on the spatial position of the examination region, in accordance with the invention the position of the examination region relative to surrounding structures is now determined in a second method step 43. In a third method step 45, the magnetic field gradients are also selected dependent on the position of the examination region relative to the surrounding structures. In this manner, the selection of the magnetic field gradients takes into account features that cause nuclear spins of the surrounding structures not to also be excited as well, by avoiding the situation in which their chemical shift causes their excitation region not to coincide with the examination region. This is explained in detail in the following using FIG. 3.

FIG. 3 shows three two-dimensional overview images 51 of a phantom 53, which two-dimensional overview images 51 are orthogonal to one another. The phantom 53 has a spherical central region 55 that is surrounded by a fatty substance 57. The central region 55 contains substances (among others N-acetyl-aspartate, creatine, choline, myo-inositol) whose ratios simulate the conditions existing in a human body. A cuboid examination region 59 that is to be examined by means of magnetic resonance spectroscopy lies in the central region 55. The examination region 59 is in immediate proximity to a fatty substance 57 surrounding the central region 55. When magnetic field gradients are now selected such that predominantly nuclear spins in this examination region 59 are excited to resonance, it may occur that nuclear spins of the surrounding fatty substance 57 are excited as well. This is because, due to the chemical shift, nuclear spins of the fatty substance 57 exhibit a slightly different resonance frequency than nuclear spins in the central region 55. This leads to the situation of the excitation region 61 for nuclear spins with the chemical shift of fat being spatially displaced in comparison to the examination region 59. This spatial shift depends on, among other things, the strength and the polarity of the magnetic field gradients that are used for excitation of the examination region 59.

The position of the surrounding fatty substance 57 relative to the examination region 59 is taken into account in an embodiment of the inventive method, such that magnetic field gradients used for excitation of the nuclear spins are selected to cause the displaced excitation region 65 that then arises for nuclear spins with a chemical shift of fat, to be farther removed from the surrounding fatty substance 57 than the examination region 59 itself. An excitation of nuclear spins in the surrounding fatty substance 57 is largely avoided in this manner, such that the measured (detected) signal exhibits a distinctly lesser contamination by nuclear magnetic resonances from the surrounding fatty substance 57.

Based on known anatomical relationships, the position of structures surrounding an examination region 59 relative to the examination region 59 is likewise known. In this case the magnetic field gradients can also be determined automatically, such that upon excitation of nuclear spins in the examination region 59, nuclear spins of the surrounding structure are excited as well but only in a lesser manner. For example, examinations and measurements of the brain are suitable for such an embodiment variant of the method, since here typically only slight inter-individual margins of fluctuation exist in the anatomical relationships.

In this case the selection of the magnetic field gradients can be established, for example, based on a model patient and can be stored in a data store. For implementation of an analogous examination on a patient, the selection of the magnetic field gradients is retrieved and, if applicable, adapted to the specific conditions. Segmentation algorithms, pattern recognition algorithms and registration methods can possibly be used to improve the automatic embodiment of the method in order to taken into account remaining inter-individual differences. In the simplest case, the gradients are selected such that the excited volume for resonance frequencies in the region of the fat always lies in the direction of magnetic center relative to the measurement volume.

A different embodiment of the method can predominantly be used given unforeseeable anatomical relationships such as, for example, in the case of tumor illnesses. In this case the anatomical relationships are presented to a user using overview images 51 as they are, for example, to be seen using FIG. 3. A user can now mark the examination region 59. In addition to the examination region 59, the excitation region 61 of nuclear spins with a specific chemical compound shift that would result given a specific configuration of magnetic field gradients is presented to a user. The user can now monitor whether the excitation region 61 of nuclear spins with a specific chemical shift intersects with the structures surrounding an examination region, as is the case in FIG. 3. In such a case the user can interactively intervene and modify the magnetic field gradients so that the displaced excitation region 65 thereby arising lies farther removed from the surrounding structures than the examination region 59. For example, this can occur by the user changing the polarity of the magnetic field gradients. By changing the polarity of the magnetic field gradients, the position of the excitation region relative to the examination region 59 likewise shifts.

The position of the excitation region 61, or the shifted excitation region 65 shown in FIG. 3, in comparison to the examination region 59 is presented more significantly displaced than corresponds to reality, but this allows the principle underlying the inventive method to be recognized and explained more clearly.

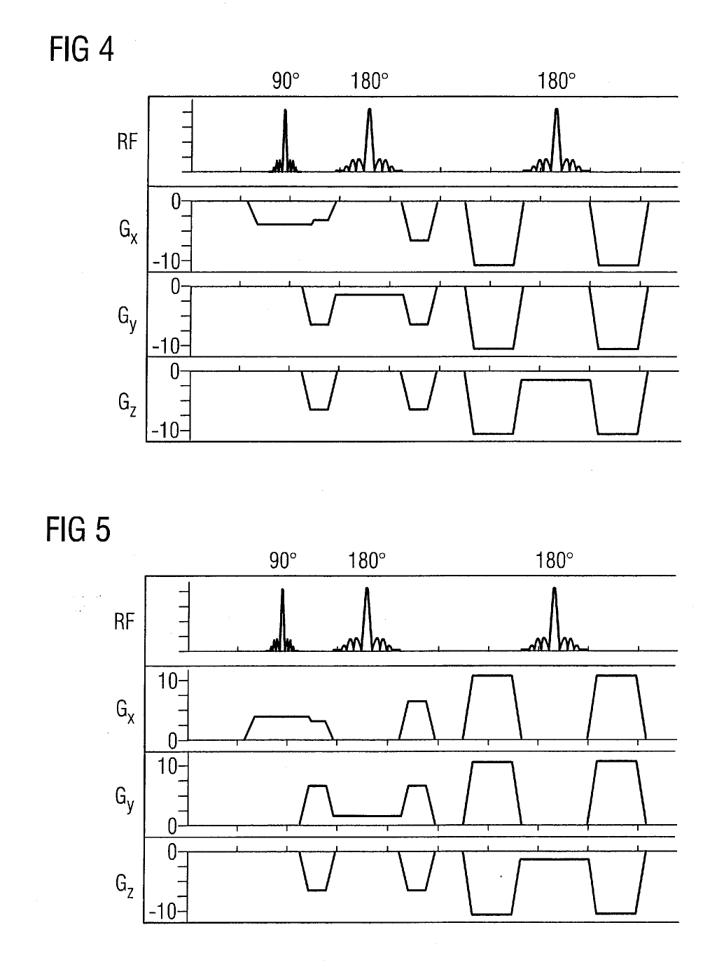

FIGS. 4 and 5 show the time curve of radio-frequency pulses RF (“radio frequency”) and magnetic field gradients that are used for selective excitation of nuclear spins in an examination region 59. In the example presented here, a PRESS sequence (“Point Resolved Spectroscopy”) is shown in which magnetic field gradients Gₓ, G_{y} and G_{z} are respectively switched in the x-, y- or, respectively, z-direction at the 90° excitation pulse or, respectively, the 180° rephasing pulses in order to excite nuclear spins in the examination region 59. The magnetic field gradients in FIG. 4 and FIG. 5 differ insofar as that the magnetic field gradients Gₓ and G_{y} in the x-direction or, respectively, y-direction are inverted, meaning that they exhibit a different polarity. Although the same examination region 59 corresponds to the magnetic field gradients Gₓ, G_{y} and G_{z} from FIG. 4 and FIG. 5, the excitation regions of nuclear spins with a specific chemical shift (for example that of fat) exhibit a different position relative to the examination region 59. Skillful selection of the polarity of the magnetic field gradients Gₓ, G_{y} and G_{z} allows the excitation region established in this manner for nuclear spins with a specific chemical shift to be directly separated from the structures surrounding the examination region 59, such that the excitation of nuclear spins in structures surrounding the examination region 59 is minimized.

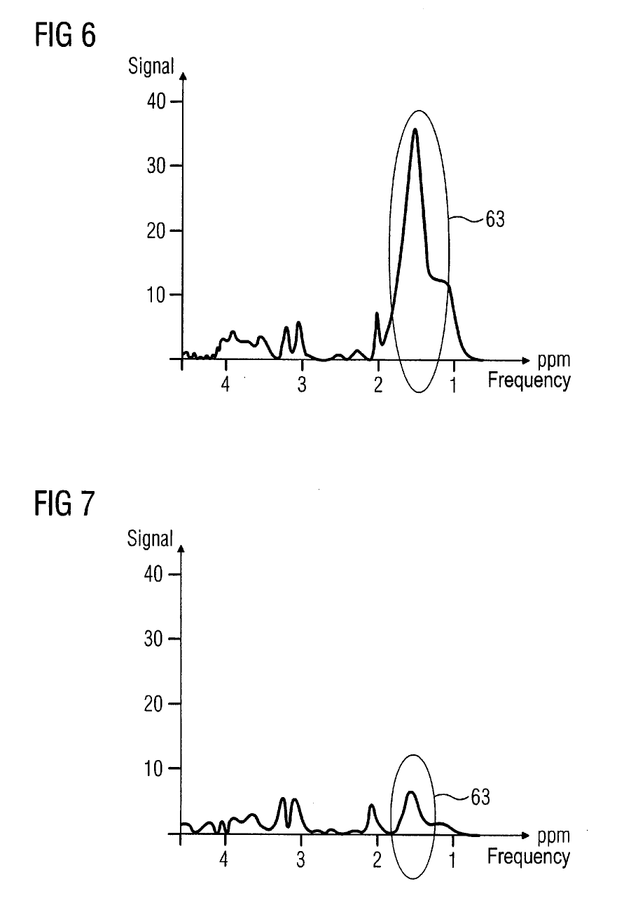

FIG. 6 and FIG. 7 respectively show the frequency spectrum of the measured signals, whereby the frequency spectra from FIG. 6 and FIG. 7 were obtained from measurement signals of an examination region 59 that has been excited with magnetic field gradients Gₓ, G_{y} and G_{z} according to FIG. 4 and FIG. 5. A spectral range 63 with a particularly high signal intensity is clearly to be recognized in FIG. 6. This signal originates from nuclear spins of the fatty substance 57 that surrounds the examination region 59 and that was excited as well together with the examination region 59 due to the chemical shift of fat. This unwanted excitation was avoided by changing the polarity of the magnetic field gradients Gₓ, G_{y} and G_{z} in FIG. 5, such that the interfering high signal intensity in the spectral range 63 is distinctly reduced in the frequency spectrum from FIG. 7. The obtained spectrum can now be evaluated in a significantly more targeted and improved manner.

Although modifications and changes may be suggested by those skilled in the art, it is the intention of the inventor to embody within the patent warranted hereon all changes and modifications as reasonably and properly come within the scope of his contribution to the art.

I claim as my invention:

1. A method for selective excitation of nuclear spins in an examination region in an examination subject comprising the steps of:
irradiating the examination subject with at least one radio-frequency excitation pulse; and
substantially contemporaneously with the irradiation of the region with said at least one radio-frequency pulse, subjecting said region to at least one slice-selective magnetic field gradient, and designing said at least one slice-selective magnetic field gradient dependent on a position of the examination region relative to at least one structure surrounding the examination region, to cause excitation of substantially only nuclear spins in said examination region to occur.

2. A method as claimed in claim 1 comprising designing said at least one slice-selective magnetic field gradient to cause an excitation region, in which nuclear spins having a resonance frequency characterized by a predetermined chemical shift, is farther from said surrounding structure than said examination region.

3. A method as claimed in claim 2 wherein said predetermined chemical shift is the chemical shift of nuclear spins in fatty tissue.

4. A method as claimed in claim 1 comprising orienting said at least one slice-selective magnetic field gradient dependent on a position of the examination region relative to said surrounding structure.

5. A method as claimed in claim 4 comprising orienting said at least one slice-selective magnetic field gradient by selecting the polarity of a magnetic field gradient generated by at least one gradient coil.

6. A method as claimed in claim 1 comprising establishing said examination region by generating an overview image of the examination subject.

7. A method as claimed in claim 1 comprising automatically designing and selecting said at least one slice-selective magnetic field gradient.

8. A method as claimed in claim 1 comprising allowing selection of said at least one slice-selective magnetic field gradient through a user interface, and selecting said at least one slice-selective magnetic field gradient by interaction of a user with said user interface.

9. A method as claimed in claim 8 comprising, while a user is interacting with said interface to select said at least one slice-selective magnetic field gradient, displaying said examination region in an overview image of the examination subject and also including display of the excitation region of nuclear spins having a resonance frequency characterized by a predetermined chemical shift.

10. A magnetic resonance apparatus comprising:
a magnetic resonance data acquisition unit, configured to interact with an examination subject therein, to acquire magnetic resonance data from an examination region of the subject;
said data acquisition unit comprising a radio-frequency system and a gradient coil system; and
a control unit that operates said radio-frequency system and said gradient coil system to acquire said magnetic resonance data, by irradiating the examination region with at least one RF pulse from said RF system to excite nuclear spins in the examination region, and substantially contemporaneously subjecting the examination subject to at least one slice-selective magnetic field gradient generated by said gradient coil system, said control unit selecting said at least one slice-selective magnetic field gradient dependent on a position of the examination region relative to at least one structure surrounding the examination region in the examination subject.
